(19) United States
(12) Patent Application Publication
(10) Pub. No.: US 2017/0321163 A1
(43) Pub. Date: Nov. 9, 2017

(54) ENZYME STABILIZERS

(71) Applicant: Henkel AG & Co. KGaA, Duesseldorf (DE)

(72) Inventors: Timothy O'Connell, Landsberg am Lech (DE); Nina Mussmann, Willich (DE); Daniela Herbst, Duesseldorf (DE); Inken Prueser, Duesseldorf (DE)

(73) Assignee: Henkel AG & Co. KGaA, Duesseldorf (DE)

(21) Appl. No.: 15/532,507

(22) PCT Filed: Nov. 17, 2015
(86) PCT No.: PCT/EP2015/076749

§ 371 Date: Jun. 2, 2017

(30) Foreign Application Priority Data
Dec. 3, 2014 (DE) 10 2014 224 748.9

(51) Int. Cl. C11D 3/386; A61K 31/4164; C11D 3/28

(52) U.S. Cl. CPC C11D 3/38663; C11D 3/38609; A61K 31/4164; C11D 3/28

(57) ABSTRACT

The present disclosure relates to detergents and cleaning agents, comprising at least one protease and at least one organic compound of formula (I), said compound acting as a protease inhibitor and thus being a suitable enzyme stabilizer, and to the use of said compounds as enzyme stabilizers in protease-containing detergents and cleaning agents. The disclosure further relates to the corresponding washing and cleaning methods and to the use of the agents described herein.

### CROSS-REFERENCE TO RELATED APPLICATION

This application is a U.S. National-Stage entry under 35 U.S.C. §371 based on International Application No. PCT/EP2015/076749, filed Nov. 17, 2015, which was published under PCT Article 21(2) and which claims priority to German Application No. 10 2014 224 748.9, filed Dec. 3, 2014, which are all hereby incorporated in their entirety by reference.

### TECHNICAL FIELD

The present disclosure relates to detergents and cleaning agents comprising at least one enzyme and at least one organic compound, which acts as a protease inhibitor and is therefore a suitable enzyme stabilizer, and to the use of said compounds in detergents and cleaning agents comprising enzymes.

### BACKGROUND

The use of enzymes in detergents and cleaning agents has for decades been established in the prior art. Commensurate with their specific activities, they serve to broaden the performance spectrum of the agent in question. Included in this context in particular are hydrolytic enzymes such as proteases, amylases, lipases and cellulases. The first three enzymes hydrolyze proteins, starches and fats and therefore contribute directly to soil removal. Cellulases are added particularly for their action on tissues. Another group of enzymes for detergents and cleaning agents is oxidative enzymes, especially oxidases, which, optionally, preferably serve to bleach stains or to generate the bleaching agent in situ in conjunction with other components. In addition to these enzymes, which are the subject of a continuous optimization, additional enzymes are continually being provided for use in detergents and cleaning agents, especially in order to be able to target specific stains in an optimal manner such as, for example pectinases, β-glucanases, mannanases or additional hemicellulases (glycosidases) for, in particular, hydrolyzing specific vegetal polymers.

The enzymes that are comprised in the longest established and in practically all modern, efficient washing and cleaning agents are proteases and, among these, serine-proteases in particular, which also include the subtilases. They serve to degrade protein-containing stains on the items to be cleaned. However, they also hydrolyze themselves (autoproteolysis) as well as all other proteins present in the agents in question, hence enzymes in particular. This takes place especially during the process of washing, thus within the aqueous wash liquor when relatively favorable reaction conditions are present. This also occurs to a lesser extent, however, during storage of the agents in question, for which reason a long storage period is always accompanied by a certain loss of protease activity as well other enzyme activity. This is particularly problematic in gel-form or liquid formulations and especially in formulations comprising water, because, in the presence of water, both the reaction medium and the hydrolysis reagent are available.

One goal in the development of detergent and cleaning agents formulations is that of stabilizing the enzymes present, particularly during storage. This is taken to mean protection against various unfavorable influences, for example against denaturation or deterioration due to physical influences or oxidation. One focus of these developments is that of protecting the proteins and/or enzymes present against proteolytic cleavage. This can be achieved by establishing physical barriers, for instance by encapsulating the enzymes within special enzyme granules or by preparing agents with two- or multi-chamber systems. The other approach, one frequently adopted, is to add chemical compounds which inhibit the proteases and thus act in general as stabilizers for proteases and for the other proteins and enzymes present. For this purpose, it is important that these be reversible protease inhibitors since the protease activity is to be inhibited only temporarily, specifically during storage, but not during the cleaning process.

Established in the prior art as reversible protease inhibitors are polyols, in particular glycerin and 1,2-propylene glycol, benzamidine hydrochloride, borax, boric acids, boronic acids or salts or esters thereof. Principally noteworthy among these are derivatives comprising aromatic groups, for instance ortho-, meta- or para-substituted phenyl boronic acids, in particular 4-formylphenylboronic acid or the salts or esters of said compounds. Particularly good protection is obtained by the use of boric acid derivatives together with polyols since they are then able to form a stabilizing complex. Also described for this purpose are peptide aldehydes, which is to say oligopeptides having a reduced C-terminus, in particular those comprising 2 to 50 monomers. Included among the peptidic reversible protease inhibitors are, among others, ovomucoid and leupeptin. Also used for this purpose are specific reversible peptide inhibitors and fusion proteins from proteases as well as specific peptide inhibitors.

Additional established enzyme stabilizers are amino alcohols such as mono-, di-, triethanol- and propanolamine and mixtures thereof, aliphatic carboxylic acids up to C12, for example succinic acid, other dicarboxylic acids, or salts of said acids. Also established for this purpose are end-capped fatty acid amide alkoxylates. Certain organic acids used as builders, as disclosed in WO 97/18287, are also able to stabilize an enzyme in addition to their function as builders.

Various classes of protease are established as detergent and cleaning agent proteases, for example metalloproteases. However, owing to their favorable enzymatic properties such as stability or optimum pH, proteases of the subtilisin type (subtilases, subtilopeptidases, EC 3.4.21.62) occupy a prominent position among the washing and cleaning agent proteases. Due to catalytically active amino acids, they are classed as serine proteases. They act as unspecific endopeptidases, meaning that they hydrolyze any acid amide bonds present within peptides or proteins. Their optimum pH usually lies well within the alkaline range. An overview of this family is provided, for example, in the article “Subtilases: “Subtilisin-like Proteases” by R. Siezen, pp. 75-95 in “Subtilisin Enzymes,” R. Bott and C. Betzel eds., New York, 1996. Subtilases are formed naturally by microorganisms; among these, the subtilisins formed and secreted by Bacillus species are the most noteworthy group within the subtilases.

Particular efforts are therefore being made to provide reversible inhibitors from precisely this class of enzymes. Due to the necessity of using high concentrations of them in this regard, polyols such as glycerol and 1,2-propylene glycol have proven to be disadvantageous because the other active substances in the agents in question may thus be present only in correspondingly smaller proportions.

Boric acid derivatives occupy a prominent position among the serine protease inhibitors effective even at comparatively low concentrations. Independently of their stabilizing effect, however, the boric acid derivatives exhibit a decisive disadvantage: many of them, for example borate, form undesirable by-products with some other detergent or cleaning agent ingredients, thus making them no longer available for the desired cleaning purpose in the agents in question, or even remaining as soiling on the material being washed.

### BRIEF SUMMARY

Compounds, detergents or cleaning agents, and methods of using compounds, detergents or cleaning agents are provided herein. In an exemplary embedment, detergents or cleaning agents include at least one protease and at least one enzyme stabilizer, wherein the at least one enzyme stabilizer is chosen from compounds of general structural formula (I)

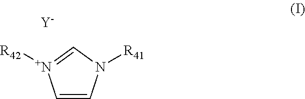

wherein R₄₁ and R₄₂, independently of one another, are unsubstituted or substituted C₁₋₆-alkyls, whereby the substituents are selected from among OH, SO₃X, NH₂, CHO, and SH;

X H is an alkali metal or ammonium; and

Y⁻ is any anion.

In another exemplary embodiment, a method includes using a compound of general structural formula (I)

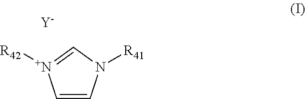

wherein R₄₁ and R₄₂, independently of one another, are unsubstituted or substituted C₁₋₆-alkyls, wherein the substituents are selected from among OH, SO₃X, NH₂, CHO and SH;

X H is an alkali metal or ammonium; and

Y⁻ is any anion;

for stabilizing an enzyme in a protease-containing detergent or cleaning agent.

In another exemplary embodiment, a compound is of a general structural formula (I)

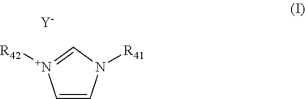

wherein R₄₁ and R₄₂, independently of one another, are unsubstituted or substituted C₁₋₆-alkyls, wherein the substituents are selected from among OH, SO₃X, NH₂, CHO and SH;

X H is an alkali metal or ammonium; and

Y− is any anion;

for use in the treatment of diseases chosen from the group consisting of respiratory diseases, inflammatory diseases, HIV, hepatitis, parasitic infectious diseases, malaria, Chagas disease, and cancer.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Therefore, the object of the present disclosure has been to identify boron-free compounds which act as protease inhibitors and are suitable for use as enzyme stabilizers in detergents and cleaning agents. Of particular interest in this regard, therefore, was the use of liquid, gel-like or paste-like detergents and cleaning agents, especially those comprising water.

This problem is solved by detergents or cleaning agents which comprise at least one protease and at least one enzyme stabilizer, whereby the at least one enzyme stabilizer is chosen from compounds of general structural formula (I)

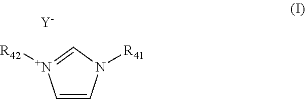

wherein R₄₁ and R₄₂, independently of one another, are unsubstituted or substituted C₁₋₆-alkyls, whereby the substituents are selected from among OH, SO₃X, NH₂, CHO and SH, and R₄₁ and R₄₂ are in particular methyl or ethyl;
X H is an alkali metal or ammonium; and
Y⁻ is any anion.

In the compounds of formula (I), R41 and R42 are in particular methyl or ethyl, quite preferably R41 being methyl and R42 ethyl, or vice versa.

In all of the aforementioned compounds, X H is an alkali metal or an alkaline earth metal, in particular sodium, potassium, calcium, magnesium or ammonium. As contemplated herein, they are preferably free salts or sodium salts.

Y is any anion and is, for example, selected from but not restricted to inorganic anions such as fluoride, chloride, bromide, sulfate, phosphate, or organic anions such as acetate, benzoate, citrate. If multivalent anions such as sulfate are used, it is obvious that the compound of formula (I) and the anion will be used in stoichiometry resulting in a balanced charge.

Without being restricted to it, one exemplary compound of formula (I) is: 1-ethyl-3-methylimidazolium acetate.

As contemplated herein, the term detergent or cleaning agent is to be understood as all agents that are suitable for the washing or cleaning of, in particular, textiles and/or solid surfaces. Additional suitable ingredients are described in detail below.

As contemplated herein, the term protease is to be understood as all enzymes that are capable of hydrolyzing acid amide links in proteins. The proteases are also described in detail below.

Without wishing to be bound by one theory, it is assumed as contemplated herein that the compounds of relevance to the disclosure form a complex with the protease to be inhibited/stabilized as contemplated herein. It is probably the case that the compound of relevance to the disclosure is inserted into the substrate-binding pocket of the protease and bonded there non-covalently. In this way, the active center of the protease is blocked by a compound which cannot be hydrolyzed by this enzyme, and it is not available to hydrolyze additional proteins that are present. This is a reversible bond, hence an equilibrium between association and dissociation. The equilibrium coefficient for this reaction is referred to as the inhibition constant or K.

The first advantage of the compounds of relevance to the disclosure over the prior art consists in the fact that they have favorable inhibition constants with respect to the proteases usable in detergents and cleaning agents. The inhibitors thus bind reversibly, meaning that they enter into temporary interactions with the enzyme that are neither too firm nor too loose. Advantageously, the majority of the protease relevant to the disclosure is thus present during storage in the form of a protease inhibitor complex. The protease and, optionally, any further proteins comprised, in particular additional enzymes, are thus protected (stabilized against proteolysis) by this enzyme. On the other hand, at the moment when the agent as contemplated herein is diluted with water to produce an aqueous washing or cleaning liquor during the cleaning process, the bond equilibrium is shifted towards dissociation so that the complex breaks down and the majority of the protease relevant to the disclosure can become proteolytically active.

The second advantage of the compounds of relevance to the disclosure over the prior art consists in the fact that they only comprise C, H, N, and O as elements, and are in particular free from boron. Therefore, they do not form the undesirable by-products with other washing or cleaning agent ingredients that are attributable to boron.

Furthermore, particularly owing to the imidazolium groups comprised, they have good solubility in water, so they can be readily incorporated into appropriate agents, and precipitation during storage is avoided.

In principle, therefore, it is assumed that the aforementioned compounds act as reversible inhibitors because they are structurally adapted to the conditions of the binding pocket in a manner similar to the substrate of the proteases. This is particularly true of serine proteases, as has been demonstrated on the basis of the examples of the present application given the positive action of the compounds experimentally described there on the basis of serine proteases, specifically subtilases, more specifically still subtilisins.

Additional objects of the present disclosure relate to:
the use of a compound described above as a reversible inhibitor and/or a stabilizer of a protease within the context of a detergent or cleaning agent formulation;
washing or cleaning methods in which a protease is made to work, which uses a compound described above for inhibiting and/or stabilizing;
the use of a washing or cleaning agent as contemplated hereinas contemplated herein for the washing and/or cleaning of textiles and/or hard surfaces; as well as
the use of a protease and a connection described above for the production of a detergent or cleaning agent.
the use of a compound described above in the treatment of diseases, for example respiratory diseases, inflammatory diseases, HIV, hepatitis, parasitic infectious diseases, malaria, Chagas disease, and cancer.

Regarding detergents or cleaning agents as contemplated herein, in one embodiment of which they are present in predominantly solid form, and in another embodiment of which they are present in predominantly liquid, paste-like or gel-like form, the enzyme, hence the protease, is comprised therein in a quantity of from about 0.05 to about 5% by weight and from about 0.05 to about 2% by weight, and the enzyme stabilizer is comprised in a quantity of from about 0.05 to about 15% by weight, from about 0.05 to about 5% by weight based on the total weight of the detergent or cleaning agent.

In various embodiments, the enzyme and the enzyme stabilizer may be present in a pre-formulated enzyme composition, whereby the enzyme is comprised in the enzyme composition in a quantity of from about 0.05 to about 15% by weight and from about 0.05 to about 5% by weight, and the enzyme stabilizer is comprised in a quantity of from about 0.05 to about 35% by weight, from about 0.05 to about 10% by weight based on the total weight of the detergent or cleaning agent. This enzyme composition, which is likewise a component of the present disclosure, can then be used in detergents or cleaning agents as contemplated herein, namely in amounts achieving the final concentrations in the detergent or detergent as specified above.

In addition to the enzyme stabilizer in accordance with the general formula specified above, an agent as contemplated herein may contain at least one additional stabilizer, in particular a polyol, such as glycerin or 1,2-ethylene glycol, and/or an antioxidant.

The protease stabilized or reversibly inhibited as contemplated herein is preferably a serine protease, in particular a subtilase, and quite preferably a subtilisin. The subtilisin can for this purpose be a wild-type enzyme or a subtilisin variant, whereby the wild-type enzyme or the parent of the variant enzyme is preferably chosen from among the following:
the alkaline protease from Bacillus amyloliquefaciens (BPN′),
the alkaline protease from Bacillus licheniformis (subtilisin Carlsberg),
the alkaline protease PB92,
subtilisin 147 and/or 309 (savinase)
the alkaline protease from Bacillus lentus, preferably from Bacillus lentus (DSM 5483),
the alkaline protease from Bacillus alcalophilus (DSM 11233),
the alkaline protease from Bacillus gibsonii (DSM 14391) or an alkaline protease at least as much as about 70% identical thereto,
the alkaline protease from Bacillus sp. (DSM 14390) or an alkaline protease at least as much as about 98.5% identical thereto, and
the alkaline protease from Bacillus sp. (DSM 14392) or an alkaline protease at least as much as about 98.1% identical thereto.

Agents as contemplated herein may comprise one or more further enzymes in addition to the protease, particularly from the following group: one or more further proteases, amylases, hemicellulases, cellulases, lipases and oxidoreductases.

The amylase(s) preferably concern an α-amylase. The hemicellulase is preferably a β-glucanase, a pectinase, a pullulanase and/or a mannanase. The cellulase is preferably a cellulase mixture or a single-component cellulase, preferably or predominantly an endoglucanase and/or a cellobiohydrolase. The oxidoreductase is preferably an oxidase, in particular a choline-oxidase, or a perhydrolase.

The agents described herein include all conceivable types of detergents or cleaning agents used in either concentrated or undiluted form for use on a commercial scale, in a washing machine or for washing or cleaning by hand. These include, for example, detergents for textiles, carpets or natural fibers with regard to which the term detergent is used. Included, for example, are dishwashing detergents for dishwashers or manual dishwashing detergents or agents for cleaning hard surfaces such as metal, glass, porcelain, ceramics, tile, stone, coated surfaces, plastics, wood or leather, in regard to which the term detergent is used, thus also including both manual and machine dishwashing detergents as well as scouring agents, glass cleaners, scented toilet cleaning products, etc. Additionally included among detergents and cleaning agents in the context of the disclosure are washing aids, a dose of which is added to the actual detergent during manual or machine textile washing in order to achieve an additional effect. Detergents and cleaning agents furthermore included in the context of the disclosure are agents for textile pre- and post-treatment, meaning those brought into contact with the actual items for washing prior to the washing process, for example to dissolve stubborn soils, as well as agents used in a subsequent step for providing the washed items with additional desirable properties such as a pleasant feel, resistance to wrinkles or a low static charge. Counted among the latter agents are, among others, fabric softeners.

Embodiments of the present disclosure include all solid, powder, liquid, gel-like or paste-like dosing forms of the agents described herein, which may optionally serve for multiple phases and can be present in either a compressed or an uncompressed form. The agent can be present as a granulated powder, in particular having a bulk weight from about 300 g/L to about 1,200 g/L, in particular from about 500 g/L to about 900 g/L, or from about 600 g/L to about 850 g/L. Included among the dosing forms for the agent as contemplated herein are extruded products, granules, tablets, or pouches. Alternatively, the agent can also be liquid, gel-like or paste-like, for example in the form of a non-aqueous liquid detergent or dishwashing liquid or a non-aqueous paste, or in the form of an aqueous liquid detergent or dishwashing agent, or a water-containing paste. Furthermore, the agent can be present as a one-component system. An agent of this kind consists of one phase. Alternatively, an agent can also consist of several phases. Such an agent is thus divided into several components.

The detergents or cleaning agents described herein, which can be present as powdered solids, in recompacted particle form, as homogeneous solutions, or as suspensions, may furthermore comprise all well-known and typical ingredients found in detergents and cleaning agents of this kind, whereby at least one additional ingredient is preferably present in the detergent or cleaning agent. The agents described herein can in particular comprise surfactants, builders (structural materials), bleaches or bleach activators. They may further comprise water-miscible organic solvents, sequestrants, electrolytes, pH regulators and/or additional auxiliaries, such as optical brighteners, graying inhibitors, foam regulators, and colorants and fragrances, as well as combinations thereof.

Advantageous ingredients of the agents described herein are disclosed in international Patent Application WO2009/121725, beginning on p. 5, second to last paragraph, and ending on p. 13 after the second paragraph. Explicit reference is made to this disclosure, and the disclosure content therein is incorporated into the present patent application.

A further aspect of the present disclosure is a method for cleaning textiles or hard surfaces, wherein at least one method step uses an agent described herein.

Such methods include manual as well as machine methods, whereby machine methods are preferable. In general, methods for cleaning textiles are distinguished by the fact that various active cleaning substances are applied to the item for washing, and, after the time of action, are washed off, or that the item for washing is treated in some other way with a detergent or a solution or dilution of said agent. The same applies to methods for the cleaning of all materials other than textiles, hard surfaces in particular. At least one of the steps of any conceivable washing or cleaning method can be enhanced through the application of a detergent or cleaning agent as described herein, thereby constituting embodiments of the present disclosure.

An additional object of the present disclosure is the use of an agent described herein for the cleaning or washing of textiles or the cleaning of hard surfaces.

Yet another object of the disclosure is the use of the compounds described herein for stabilizing an enzyme in a protease-containing detergent or cleaning agent.

Finally, a further object of the disclosure is the use of a compound described above of the structural formula (I) in the treatment of illnesses such as respiratory diseases, inflammatory diseases, HIV, hepatitis, parasitic infectious diseases, malaria, Chagas disease, and cancer, or the use of the compounds described herein (i) as a medication, or (ii) in the treatment of the illness specified above.

All facts, subject-matter and embodiments described in regard to the agent described herein are also applicable to the methods and uses specified above. Therefore, reference is expressly made at this juncture to the disclosure made at the appropriate place with the instruction that said disclosure also applies to the methods and uses described above.

### EXAMPLES

### Example 1

The storage stability of protease-containing detergents and cleaning agents was tested for use as enzyme stabilizers in the presence of candidate compounds. For this purpose, the candidate compounds were formulated in the presence of up to 1% (w/w) 1,2-propanediol in a detergent or cleaning agent formulation (see Table 2). The protease (1% PUR) was then added to the formulation, and the formulation stored for 8 weeks at 30° C.

In parallel batches, the proteolytic activity of 1% protease was determined in 4% PG (1,2-propanediol), 85% matrix (see Table 1), then, each following incubation with 1% of the candidate compound (specified in w/w; QS 100% with water) determined in half of the batches in the formulation as indicated in Table 1 by the release of the chromophore para-nitroaniline from the succinyl-alanine-alanine-proline-phenylalanine para-nitroanilide substrate (AAPF-pNA; Bachem L-1400). The release of the pNA caused an increase in the absorbance at 410 nm, the progress over time of which is a measure of enzymatic activity. The measurement was carried out at a temperature of 25° C. at pH 8.6 and a wavelength of 410 nm. The time of measurement was 5 min at a measurement interval of from 20 to 60 seconds.

For evaluation, the initial values for the proteolytic activity of the agent in question were compared to the values determined after storage. The higher the residual activity is after storage, the better the inactivation of the protease present is during storage, and the better the compound in question is as a stabilizer as contemplated herein. The stabilizing effect of each tested compound is thus measured as a relative percentage increase in the residual protease activity.

TABLE 1
Formulation for activity assay

| Component | Quantity (% by weight) |
|---|---|
| Demineralized water | Residual |
| Citric acid | 1.42623 |
| Defoamer (10%) | 0.008 |
| Fatty alcohol ether sulfate | 5.6 |
| (70%) | |
| Fatty alcohol ether | 4.4 |
| Alkylbenzene sulfonic acid | 4.4 |
| (96%) | |
| C₁₂₋₁₈ fatty acid | 2.4 |
| NaOH (50%) | 0.95 |
| Glycerin | 2 |
| Phosphate (DTPMP-Na₇H₃) | 0.2 |
| (32%) | |
| Preservative | 0.10 |
| Ethanol (93%) | 1 |

The pH value of the formulation was adjusted to 8.4 by the NaOH. The formulation was clear and without color.

1-ethyl-3-methylimidazolium acetate was tested as a candidate compound.

TABLE 2
Measured residual protease activity of the candidate compound

| | Relative residual activity |
|---|---|
| Candidate compound (KV) | after incubation with KV |
| 1-ethyl-3-methylimidazolium acetate | 122% |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Detergents or cleaning agents which comprise at least one protease and at least one enzyme stabilizer, wherein the at least one enzyme stabilizer is chosen from compounds of general structural formula (I) 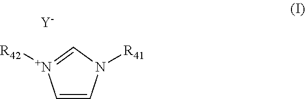
wherein R₄₁ and R₄₂, independently of one another, are unsubstituted or substituted C₁₋₆-alkyls, whereby the substituents are selected from among OH, SO₃X, NH₂, CHO, and SH;
X H is an alkali metal or ammonium; and
Y⁻ is any anion.

2. Detergents or cleaning agents according to claim 1, wherein R₄₁ and R₄₂ are methyl or ethyl.

3. Detergents or cleaning agents according to claim 1, wherein the at least one enzyme stabilizer is 1-ethyl-3-methylimidazolium acetate.

4. Detergents or cleaning agents according to claim 1, wherein the at least one protease is contained in a quantity of from about 0.05- to about 5% by weight, and the enzyme stabilizer is contained in a quantity of from about 0.05- to about 15% by weight based on the total weight of the detergent or cleaning agent.

5. (canceled)

6. A method for washing textiles or cleaning hard surfaces, wherein a solid detergent or cleaning agent according to claim 1, is used in at least one method step.

7. A method comprising using a compound of general structural formula (I) 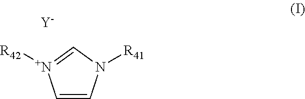
wherein R₄₁ and R₄₂, independently of one another, are unsubstituted or substituted C₁₋₆-alkyls, wherein the substituents are selected from among OH, SO₃X, NH₂, CHO and SH;
X H is an alkali metal or ammonium; and
Y⁻ is any anion;
for stabilizing an enzyme in a protease-containing detergent or cleaning agent.

8. A compound of general structural formula (I) 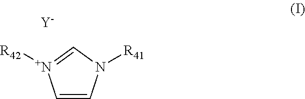
wherein R₄₁ and R₄₂, independently of one another, are unsubstituted or substituted C₁₋₆-alkyls, wherein the substituents are selected from among OH, SO₃X, NH₂, CHO and SH;
X H is an alkali metal or ammonium; and
Y− is any anion;
for use in the treatment of diseases chosen from the group consisting of respiratory diseases, inflammatory diseases, HIV, hepatitis, parasitic infectious diseases, malaria, Chagas disease, and cancer.

9. Detergents or cleaning agents according to claim 1, wherein R₄₁ is methyl and R₄₂ is ethyl.

10. Detergents or cleaning agents according to claim 1, wherein R₄₁ is ethyl and R₄₂ is methyl.

11. Detergents or cleaning agents according to claim 1, wherein the at least one protease is contained in a quantity of from about 0.05 to about 5% by weight based on the total weight of the detergent or cleaning agent.

12. Detergents or cleaning agents according to claim 1, wherein the at least one protease is contained in a quantity of from about 0.05 to about 2% by weight based on the total weight of the detergent or cleaning agent.

13. Detergents or cleaning agents according to claim 1, wherein the enzyme stabilizer is contained in a quantity of from about 0.05 to about 15% by weight based on the total weight of the detergent or cleaning agent.

14. Detergents or cleaning agents according to claim 1, wherein the enzyme stabilizer is contained in a quantity of from about 0.05 to about 5% by weight based on the total weight of the detergent or cleaning agent.

15. Detergents or cleaning agents according to claim 1, wherein the at least one protease is contained in a quantity of from about 0.05 to about 2% by weight, and the enzyme stabilizer is contained in a quantity of from about 0.05 to about 5% by weight based on the total weight of the detergent or cleaning agent.
